(19) United States
(12) Patent Application Publication
(10) Pub. No.: US 2013/0012799 A1
(43) Pub. Date: Jan. 10, 2013

(54) PORTABLE TERMINAL DEVICE AND BIOLOGICAL INFORMATION ACQUISITION METHOD

(75) Inventors: Yasuhiro Sasaki, Tokyo (JP); Hiroshi Sakai, Tokyo (JP); Masatake Takahashi, Tokyo (JP); Shigeki Shinoda, Tokyo (JP)

(73) Assignee: NEC CORPORATION, Tokyo (JP)

(21) Appl. No.: 13/636,729

(22) PCT Filed: Mar. 1, 2011
(86) PCT No.: PCT/JP2011/054662

§ 371 Date: Sep. 24, 2012

(30) Foreign Application Priority Data
Mar. 29, 2010 (JP) 2010-075965

(51) Int. Cl. A61B 5/04

(52) U.S. Cl. 600/372

(57) ABSTRACT

A portable terminal device comprises a case 110, oscillator 171, passive element 172, voltage measurement circuit 173 and controller 180. The case 110 comprises an electrode having a surface exposed to the outside, on at least a part of the case 110. The oscillator 171 outputs an A.C. voltage signal. One end of the passive element 172 is connected to the oscillator 171, and the other end is connected to the electrode. The voltage measurement circuit 173 is connected to both ends of the passive element 172, and detects the voltage applied to the passive element 172. The control unit 180 acquires the biological information of a living body based on the voltage detected by the voltage measurement circuit 173 in a state in which an A.C. output from the oscillator 171 and the electrode exposed to the outside is in contact with the living body.

### TECHNICAL FIELD

The present invention relates to a portable terminal device comprising a function for acquiring biological information and a biological information acquisition method.

### BACKGROUND ART

In recent years, under the circumstances of the increased focus on health, a portable terminal is proposed with increasing an added value by providing a function for understanding the state of health. For example, the portable telephone device referred to in Patent Literature 1 comprises a CCD camera which photographs the surface of a hand to which the CCD camera is placed closely, and functions as a moisture content sensor by calculating the moisture content from the photographic image. In addition, the portable telephone device referred to in Patent Literature 2 functions as a body fat meter, by measuring the body fat with plural electrodes provided in the portable telephone device.

### PRIOR ART LITERATURE

### Patent Literature

Patent Literature 1: Unexamined Japanese Patent Application Kokai No. 2006-098098

Patent Literature 2: Unexamined Japanese Patent Application Kokai No. 2000-229072

### DISCLOSURE OF INVENTION

### Problem to be Solved by the Invention

However, when acquiring the biological information of the user by measuring the moisture content in the skin or body fat, with the portable telephone described in Patent Literature 1 and Patent Literature 2, the user needs to take a specific active action for measurement. For example, when measuring the moisture content of the skin with the portable telephone device referred to in Patent Literature 1, the user needs to place the palm of the hand closely in front of the camera mounted on the portable telephone device. In addition, when measuring the body fat with the portable telephone referred to in Patent Literature 2, the user must place his own fingers in contact with the 4 electrodes provided in the portable telephone device.

The present invention has been made in view of the foregoing problems, and it is an objective of the present invention to provide a portable terminal device and biological information acquisition method capable of acquiring the biological information of the user, even if no active action is taken by the user to acquire the biological information.

### Means for Solving the Problem

In order to achieve the aforementioned objective, a portable terminal device of the present invention, comprises a case that comprises an electrode which has a surface exposed to the outside, on at least a part of the case. The case includes: an oscillator which outputs an A.C. voltage signal; a passive element, one end of which is connected to the oscillator, and the other end of which is connected to the electrode; a voltage measurement circuit which detects the voltage applied to the passive element by being connected to both ends of a passive element; and a controller which acquires the biological information of a living body, based on a measured voltage value comprising the voltage detected by the voltage measurement circuit in a state in which an A.C. voltage signal is output from the oscillator, and in which the living body is in contact with the surface exposed to the outside of the electrode.

In order to achieve the aforementioned object, the biological information acquisition method of the present invention executed with a portable terminal device, comprising: an A.C. voltage output step of outputting A.C. voltage from an oscillator; a voltage detection step of detecting a voltage applied to a passive element by the A.C. voltage output in the A.C. voltage output step, wherein one end of the passive element is connected to the oscillator, and the other end is connected to an electrode in a state in which a living body makes contact with the electrode which has a surface exposed to the outside, the electrode which is provided on at least a part of a case of the portable terminal device; and a biological information acquisition step of acquiring the biological information of a living body based on the voltage detected in the voltage detection step.

### Effects of the Invention

According to the present invention, even if there is no active action by the user to acquire the biological information, a portable terminal device and its biological information acquisition method can be provided which is capable of acquiring the biological information of the user.

### BRIEF DESCRIPTION OF DRAWINGS

FIG. 1A is a front view of the portable telephone according to Embodiment 1 of the present invention;

FIG. 1B is a rear view of the portable telephone according to Embodiment 1 of the present invention;

FIG. 2 is a general exploded perspective view of the portable telephone according to Embodiment 1 of the present invention;

FIG. 3 is a cross-section along the cutting plane line A-A of FIG. 1A;

FIG. 4 is a functional block diagram of the portable telephone according to Embodiment 1 of the present invention;

FIG. 5 is a diagram showing an example of the circuit configuration of a biological information detector;

FIG. 6 is a flow chart explaining the biological information acquisition process to be executed by the controller;

FIG. 7 is a diagram explaining a method for acquiring the body fat percentage and the sweat rate with reference to the biological information DB;

FIG. 8A is a front view of the portable telephone according to Embodiment 2 of the present invention;

FIG. 8B is a rear view of the portable telephone according to Embodiment 2 of the present invention;

FIG. 9 is a general exploded perspective view of the portable telephone according to Embodiment 2 of the present invention;

FIG. 10 is a cross-section along the cutting plane line B-B of FIG. 8A;

FIG. 11A is a front view of the portable telephone according to a modified example of Embodiment 2 of the present invention;

FIG. 11B is a rear view of the portable telephone according to a modified example of Embodiment 2 of the present invention;

FIG. 12 is a general exploded perspective view of the portable telephone according to a modified example of Embodiment 2 of the present invention; and

FIG. 13 is a cross-section along the cutting plane line C-C of FIG. 11A.

### MODE FOR CARRYING OUT THE INVENTION

### Embodiment 1

An explanation of Embodiment 1 of the present invention is provided hereafter with reference to the drawings. Moreover, in Embodiment 1 of the present invention, an explanation is provided of an example of a portable telephone used as the portable terminal device capable of acquiring the biological information of the user. However, the present invention is not limited to this, and it may also, for example, be used as another type of portable terminal device, such as a PDA (Personal Digital Assistant), or a notebook personal computer or the like.

Moreover, in Embodiment 1 and Embodiment 2 below, the “biological information” is information relating to the state of the user's body, and specifically expresses data relating to the moisture content of the skin, the body fat percentage, the sweat rate and the like.

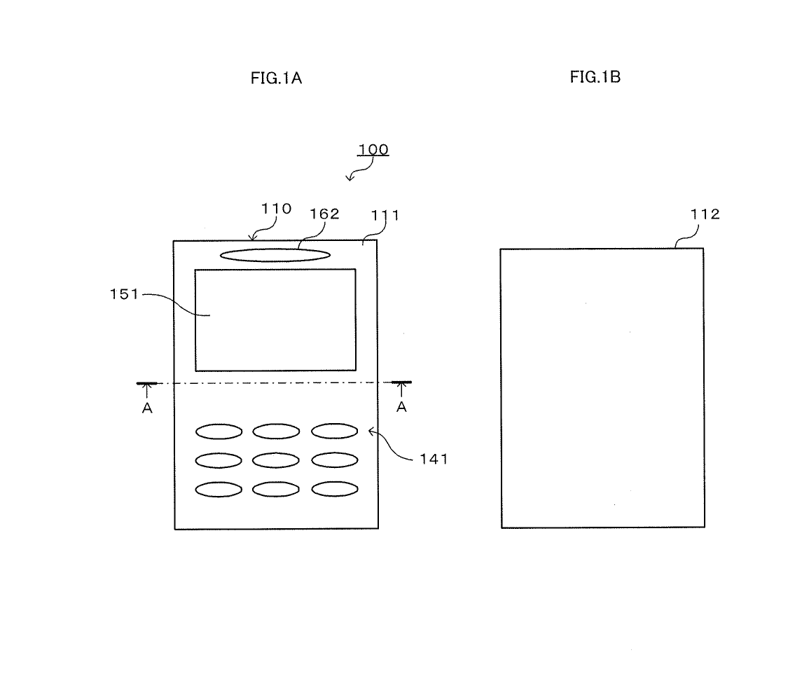

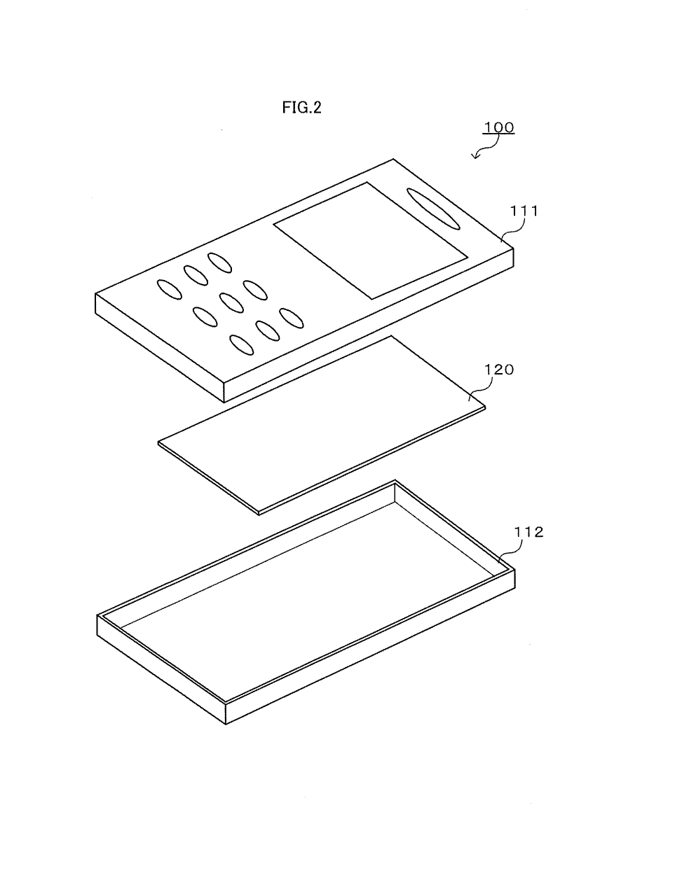

FIG. 1A and FIG. 1B are front and rear views of the portable telephone 100 respectively according to Embodiment 1 of the present invention, and FIG. 2 is a exploded perspective view thereof. The portable telephone 100 comprises a case 110, and the circuit board 120 housed within the case 110.

The case 110 houses each type of component of the portable telephone 100, and the outer surface which is the reverse of its inner surface is exposed to the outside. In addition, the case 110, in a biological information detector 170, explained hereafter, functions as an electrode. The case 110, as shown in FIG. 2, is formed in a boxlike shape with a front case 111 and a rear case 112. In addition, the case 110 is formed from electrically conducting material, such as magnesium alloy or SUS or titanium alloy. In the front case 111 is formed an opening, in order to expose a display panel 151 or hardware keys 141 or a speaker 162 or the like, explained hereafter.

On a circuit board 120 is formed an electronic circuit, on which electronic components are mounted for realizing each type of function of the portable telephone 100, explained hereafter.

Hereafter, the side on which the electronic circuit is formed, and its reverse, of the circuit board 120, are referred to as surfaces of the circuit board 120; the sides of the circuit board 120 which intersect with these surfaces are referred to as the side surfaces of the circuit board 120, and in addition, and the sides of the case 110, parallel to the above-mentioned surfaces of the circuit board 120 are referred to as the surfaces of the case 110 and the sides of the case 110 parallel to the side surfaces of the circuit board 120 are referred to as the side surfaces of the case 110.

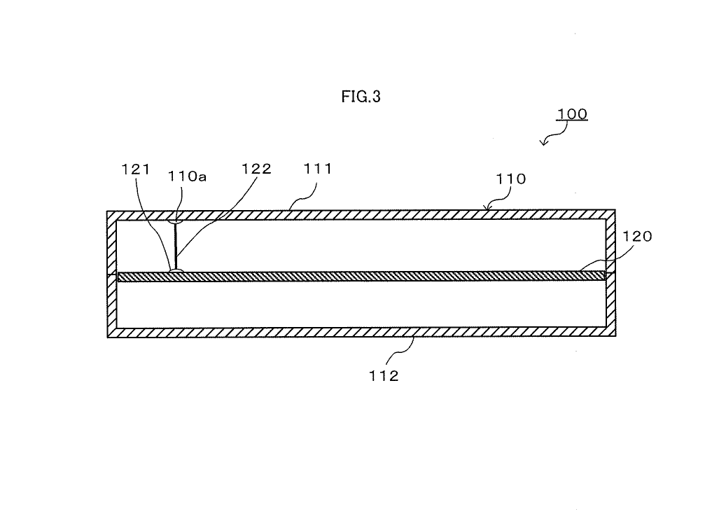

The circuit board 120 is fixedly placed within the case 110 by fixing means not shown in the figures. In addition, as shown in the cross-section in FIG. 3, the contact 121 on the circuit board 120 is connected so as to be electrically conducted with the contact 110a attached to the inside surface of the case 110 (specifically, the inner surface facing the surface of the circuit board 120, of the front case 111) through a wire 122. Furthermore, the contact 121 on the circuit board 120 and the case 110 are electrically conducted.

The positions of the contacts 121 and 110a are not limited to the positions shown in FIG. 3. For example, the contact 121 may also be situated on the surface facing the rear case 112, of the circuit board 120, or on the side surface of the circuit board 120. In addition, the contact 110a may also be situated on the inner surface facing the surface of the circuit board 120, of the rear case 112, or on the inside surface of the front case 111 or the rear case 112.

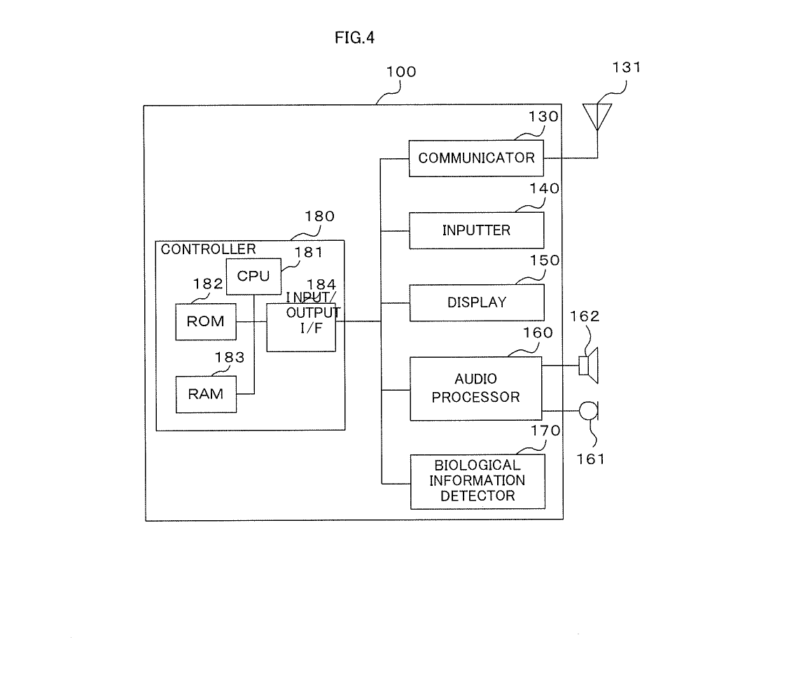

Next, An explanation is provided concerning the functions of the portable telephone 100. FIG. 4 is a block diagram showing each function of the portable telephone 100. As shown in FIG. 4, the portable telephone 100 comprises a communicator 130, an inputter 140, a display 150, an audio processor 160, a biological information detector 170, and a controller 180. Specifically, these functions are realized by the electronic circuit which comprises the electronic components and the like mounted on the circuit board 120.

The communicator 130 comprises an internal antenna 131 which transmits or receives electronic waves, and the electronic circuit (not shown in the diagram) which modulates signals to be transmitted or demodulates received signals, respectively. Data to be transmitted or received by the communicator 130 is, for example, the audio data of telephone conversations or image data or E-mail data or the like.

The inputter 140 receives the input operation of the user, and outputs the input data (input information) to the controller 180 according to the received input operation. The inputter 140, as shown, for example, in FIG. 1A comprises the plural hardware keys 141 capable of inputting figures and letters. The inputter 140 is connected to the controller 180, and the input information is stored in a ROM (Read-Only Memory) 182 of the controller 180. In the present embodiment, the input information, for example, is the body information of the height, age, gender, weight and/or the like of the user, and is used in the acquisition of the biological information in the biological information acquisition process, described hereafter.

The display 150 displays text and/or images based on the text data and/or image data output from the controller 180. The display 150, for example, comprises a drive circuit (not shown in the diagram) and the display panel 151.

The drive circuit drives the display panel 151 to display text and/or images, based on the text data and/or image data output from the controller 180. The display panel 151 comprises a liquid crystal panel or an organic EL (electroluminescence) panel or the like. In the present embodiment, the display 150 displays text and/or images expressing the biological information of the body fat percentage, the sweat rate and/or the like acquired through measurements made by processes described hereafter.

The audio processor 160 performs audio input and its encoding at the time of a telephone conversation, and decodes the audio data wirelessly received from the communicator. The audio processor 160 comprises a microphone 161 for creating audio data by performing an audio input by the user, a speaker 162 for playing back the audio data as sound and a processing circuit (not shown in the diagram) for encoding the input audio data, and for decoding the wirelessly received audio data. The speaker 162 outputs sounds for notifying the user of, for example, acquired biological information.

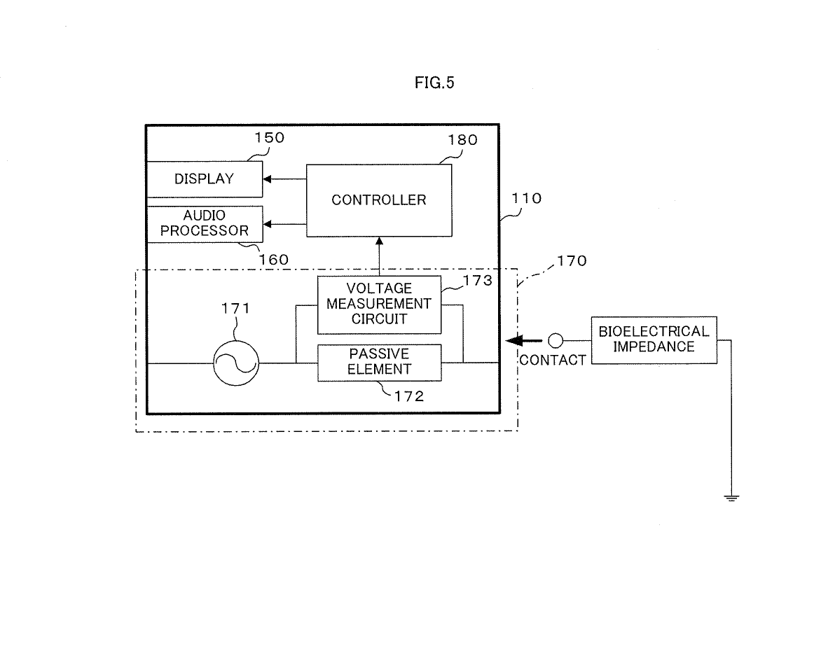

The biological information detector 170 takes measurement for acquiring the biological information of the user. An example of the circuit configuration of the biological information detector 170 is shown in FIG. 5. As shown in FIG. 5, the biological information detector 170 comprises an oscillator 171, a passive element 172, a voltage measurement circuit 173 and a case 110.

The oscillator 171 outputs an A.C. voltage signal (for example, 1 k hz, 1 V). In addition, one terminal of the oscillator 171 is electrically connected to the case 110, and the other terminal is electrically connected to the passive element.

The passive element 172 is composed to include at least one of either a resistor, a condenser, or a coil, and is electrically connected to the oscillator 171. In addition, the passive element 172 is electrically connected to the contact 121 on the circuit board 120 shown in FIG. 3, and is electrically connected to the case 110 through the contact 121.

The voltage measurement circuit 173 is electrically connected to both terminals of the passive element 172, and measures the voltage between both ends of the passive element 172. In addition, the voltage measurement circuit 173 is also electrically connected to the controller 180, and outputs the measured voltage to the controller 180 as a voltage signal.

The case 110 functions as an electrode of the biological information detector 170. The case 110, as shown in FIG. 3, comprises the contact 110a on the surface facing the surface of the circuit board 120. The contact 110a is electrically connected to the contact 121 and to the passive element 172 on the circuit board 120 through the wire 122.

In the biological information detector 170 composed as described above, in a state in which, for example, the user does not make contact with the case 110, if the A.C. voltage signal is output from the oscillator 171, then the voltage measurement circuit 173 detects the specific voltage value determined by the output A.C. voltage signal and the impedance of the passive element 172. Also, the voltage signal showing the detected voltage value is output to the controller 180, and the controller 180 stores the voltage value in the ROM 182 as the standard voltage value.

In a state in which the user is touching the case 110, if an A.C. voltage signal is output from the oscillator 171, then electric current, determined with the bioelectrical impedance of the user, flows through the case 110. In this state, the voltage measurement circuit 173 measures the voltage between both ends of the passive element 172, and outputs it to the controller 180 as a voltage signal. The controller 180 acquires the voltage value of the voltage signal by sampling, and stores it in a RAM 183. Furthermore, the controller 180 obtains the measured voltage value as a representative value from the sampled voltage value, and calculates the difference between the measured voltage value and the standard voltage value as the differential voltage value.

The calculated differential voltage value changes corresponding to the bioelectrical impedance of the user. In addition, the bioelectrical impedance is a value which changes according to the living body state, which corresponds to the biological information of the body fat percentage, the sweat rate and/or the like of the user. In other words, there is a specific relationship between the differential voltage value and the biological information.

On the other hand, from statistical procedures, the relationship between the differential voltage values and the biological information for plural individuals, each having a different age, gender, body weight and height, is obtained as correlation information, and the data of the correlation information is stored in the ROM 182.

The controller 180 comprises a CPU (Central Processing Unit) 181, a ROM (Read-Only Memory) 182, a RAM (Random Access Memory) 183, an input/output interface (I/F) 184, and the like.

The CPU 181 executes control of each type of component of the portable telephone 100 and performs each type of process. The ROM 182 stores control programs to be used by the CPU 181 to control each component, programs for each type of application software, telephone book data, electronic mail data and user information for calculating the biological information. The RAM 183 is used as a working storage area when the CPU 181 executes each type of process.

The CPU 181, using the correlation information and the body information (information of gender, age, height, and body weight) of the user stored in the ROM 182, generates, for example by an interpolation process, a relationship between the differential voltage values and the biological information for individuals whose body information conforms to the body information of the user, as calibrated correlation information. In addition, the controller 180 performs biological information acquisition processing, which acquires the biological information of the user from the calibrated correlation information, based on the differential voltage value calculated from the voltage impressed on the passive element 172, i.e., the measured voltage value, detected by the voltage measurement circuit 173 at the time when the user is touching the case 110, which functions as an electrode. In addition, the controller 180 outputs the acquired biological information to the display 150 or the audio processor 160.

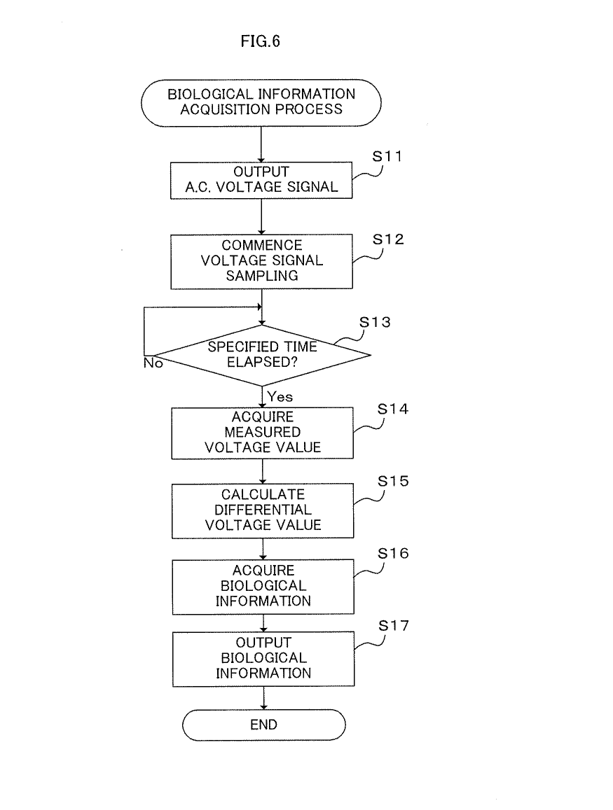

Next, an explanation is provided concerning actions of the controller 180 in the biological information acquisition process, with reference to the flow chart of FIG. 6. The controller 180 commences the biological information acquisition process with being triggered by a specific user operation. In this instance, the specific user operation is not an active user operation for acquiring the biological information, but is a user operation, the primary objective of which is other than that of acquiring the biological information. Specifically, the controller 180 commences the biological information acquisition process with being triggered by the user operation to select a button to transmit or receive Email, or to press a specific keyboard, for example, “0” key while talking on the telephone.

At the time of such an operation, since the user is generally in a state of grasping the case 110, in other words, since the user is in a state of contact with the case 110 which is the electrode of the biological information detector 170, the biological information detector 170 is able to take measurement in order to acquire the biological information of the user during the operation.

In the following explanation, the standard voltage value is pre-stored in the ROM 182. Specifically, for example, at the stage of manufacturing the portable telephone 100, in a state in which a living body is not in contact with the portable telephone 100, the controller 180 controls the oscillator 171 to output an A.C. voltage signal (for example, 1 k Hz, 1V). Also, the controller 180 controls ROM 182 to store a voltage signal which expresses the voltage value between both ends of the passive element 172, which is measured and output by the voltage measurement circuit 173 as the standard voltage value.

Once the biological information acquisition process commences, initially the controller 180 activates the oscillator 171 to output (step S11) an A.C. voltage signal (for example, 1 k Hz, 1V).

Next, the controller 180 commences (step S12) sampling of the temporally changing voltage signal between both ends of the passive element 172, the voltage signal which the voltage measurement circuit 173 measures and outputs. Also, the controller 180 controls the RAM 183 to store the sampled voltage values.

The controller 180 determines (step S13) whether a specified time (for example, 3 seconds) has elapsed, based on an internally housed timer. If the specified time is determined not to have elapsed (step S13; No), there is a wait until it has elapsed.

If it is determined that the specified time has elapsed (step S13; Yes), the controller 180 acquires (step S14) the measured voltage value which is the representative value of the voltage values stored in the RAM 183.

Here, the method which acquires the measured voltage value from the voltage values stored in the RAM 183, is as follows. For example, the arithmetic mean value is calculated, from all of the voltage values acquired by the sampling at specified times; and this arithmetic mean value may be used as the measured voltage value. Furthermore, the arithmetic mean value can be calculated from the voltage values acquired by the sampling at the specified times, with excluding the maximum value and the minimum value from the sampled voltage values, and this arithmetic mean value may be used as the measured voltage value.

Rather than the mean value, among the voltage values acquired by the sampling at the specified times, the mode value or median value may be used as the measured voltage value.

Voltage values which have a specified potential difference in comparison to the standard voltage value can be extracted from the voltage values stored in the RAM 183, and from the extracted voltage values, the representative value of, for example, the mean value or the mode value or the median value or the like can be obtained. This representative value may also be used as the measured voltage value. According to this process, for example, if within a specified time in step S13, the state between the user and the case 110 temporarily changes to the noncontact state from the contact state, the measured voltage value can be set with excluding voltage values measured under the noncontact state (which are the same as the standard voltage value). Accordingly, a measured voltage value can be obtained with high reliability with which the voltage had been measured under the state of contact.

Next, the controller 180 calculates (step S15) the difference between the measured voltage value acquired in step S14 and the standard voltage value stored in the ROM 182, as the differential voltage value.

Next, the controller 180 acquires (step S16) the biological information corresponding to the differential voltage value calculated in step S15 from the relationship (correlation information) between the pre-obtained differential voltage value and the biological information.

Here, a detailed explanation is provided on a method for acquiring the biological information corresponding to the differential voltage value from the correlation information obtained in advance.

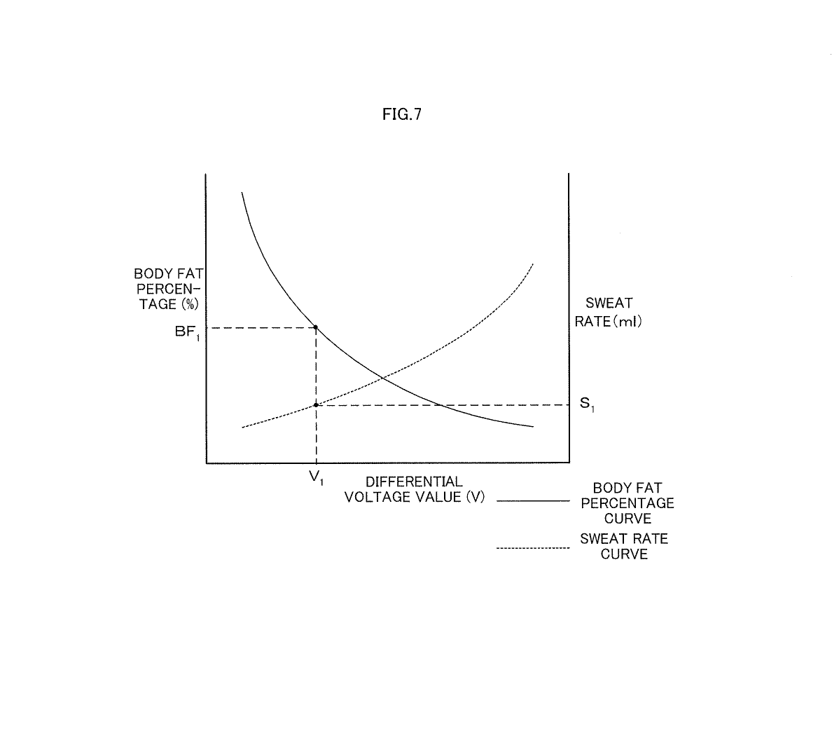

The correlation information is obtained in advance using statistical procedures, for example, as the biological information which is a function of the differential voltage value. Therefore, the controller 180, from the function obtained in advance, is able to acquire the biological information corresponding to the differential voltage value calculated in step S15. Specifically, the portable telephone 100 is able to acquire, for example, the body fat percentage and the sweat rate as the biological information. Here, the relations of the body fat percentage and the sweat rate to the differential voltage value are expressed by a body fat percentage curve and a sweat rate curve respectively, as shown in FIG. 7. From these curves, the controller 180 acquires BF₁ (%) as the body fat percentage and S₁(ml) as the sweat rate, when the differential voltage value calculated in step S15 is V₁.

The correlation information may also be expressed, such as, in the form of the plural differential voltage values and the value of the biological information corresponding to each differential voltage value, for example, using a statistical method. Specifically, for example, the controller 180 stores, in the ROM 182 as the database, the differential voltage values and the body fat percentages, associated with each other, as well as the differential voltage values and the sweat rates, associated with each other. Also, the body fat percentage and the sweat rate corresponding to the calculated differential voltage value is acquired from the values stored in the database, using, for example, the interpolation methods of linear interpolation or polynomial interpolation.

Furthermore, correlation information may be expressed by plural functions which comprise the function for each type of the biological information, such as, for example, gender, age, height and body weight, and stored in the ROM 182. The correlation information may also be stored in the ROM 182 in the form of the plural databases which comprise the database for each type of the biological information. In such a case, the biological information can be acquired more conveniently and accurately by using the function or database corresponding to the body information of the user so as to acquire the biological information corresponding to the differential voltage value.

Next, the controller 180 outputs (step S17) the biological information acquired in step S16 to the display 150 or the audio processor 160. Specifically, in step S16 for example, in the case where BF1 (%) and S1 (ml) are acquired as the body fat percentage and the sweat rate respectively, the controller 180 outputs the acquired values to the display 150, and displays an image expressing the values on the display panel 151. And then, the controller 180 terminates the biological information acquisition process.

With the portable telephone 100 composed as described above, according to Embodiment 1, the whole of the case 110 formed from an electroconductive material is used as the electrode of the biological information detector 170. If the user operates the portable telephone 100, the case 110 is grasped by and makes contact with the user. As a result, the portable telephone 100 can acquire the biological information of the user, by the user operation of the portable telephone 100, even if the primary essential objective of the operation is for the transmission/reception of e-mail or making phone calls or the like, the primary essential objective of which is not for the acquisition of biological information. In other words, the portable telephone 100 is able to acquire the biological information of the user, regardless of the active user action for acquiring the biological information.

In addition, since the whole of the case 110 is formed of electroconductive material, the position of the contact 110a may be located anywhere on the case 110. Accordingly, there is a lot of flexibility in the arrangement of the contact 121 on the circuit board 120, for the contact with the case 110. As a result, there is also a lot of flexibility in the design of the circuit board 120.

Furthermore, in measurement taken at the time of acquiring the biological information, since the whole of the case 110 functions as an electrode, the portable telephone 100 is able to acquire the biological information of the user no matter which part of the case 110 is held by the user.

Furthermore, since the whole of the case 110 functions as an electrode, it is possible to realize the large area of contact between the electrode and the user. Accordingly, in the region of contact between the user and the case 110, for example, in the palm of the hand of the user, for example, where there is a mixture of both moist and dry regions, it is possible to acquire a measured voltage value reflecting the state of a broader region of the user's palm, than when the case 110 possesses an electrode in only a part of the region of the case 110. In other words, measurement can be taken, which reflects the biological state better. As a result, the biological information can be acquired with higher accuracy.

### Embodiment 2

An explanation of Embodiment 2 of the present invention is provided hereafter, with reference to the drawings. In aforementioned Embodiment 1, the whole of the case 110 is formed from electroconductive material; however, the case 110 according to Embodiment 2 is characterized in that only a part of the case 110 is formed from electroconductive material. A detailed explanation of the case 110 comprising the characteristic part of Embodiment 2 is provided hereafter. Parts which have the same function and configuration as those of Embodiment 1 use the same labelings as those used in Embodiment 1, and a detailed explanation is omitted.

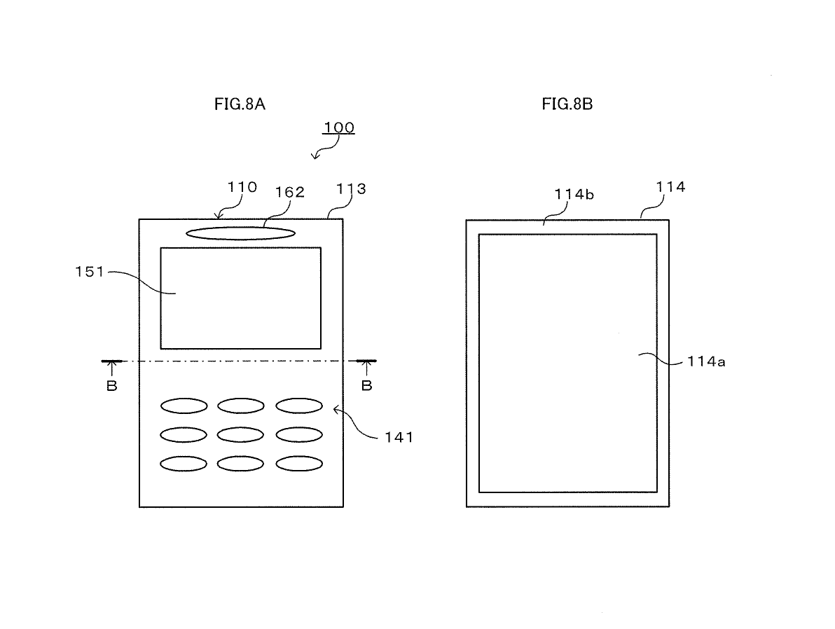

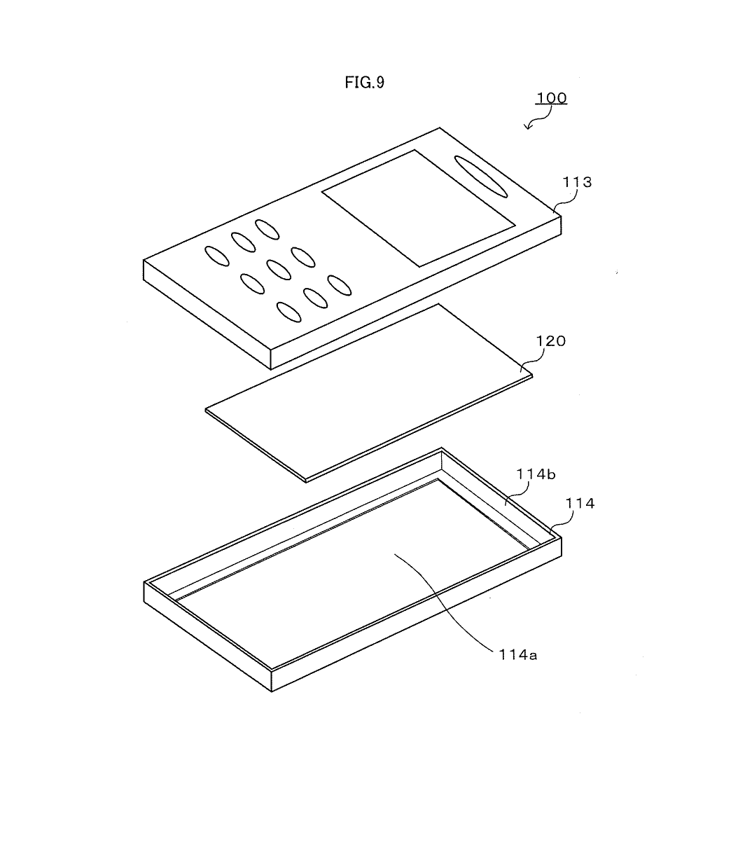

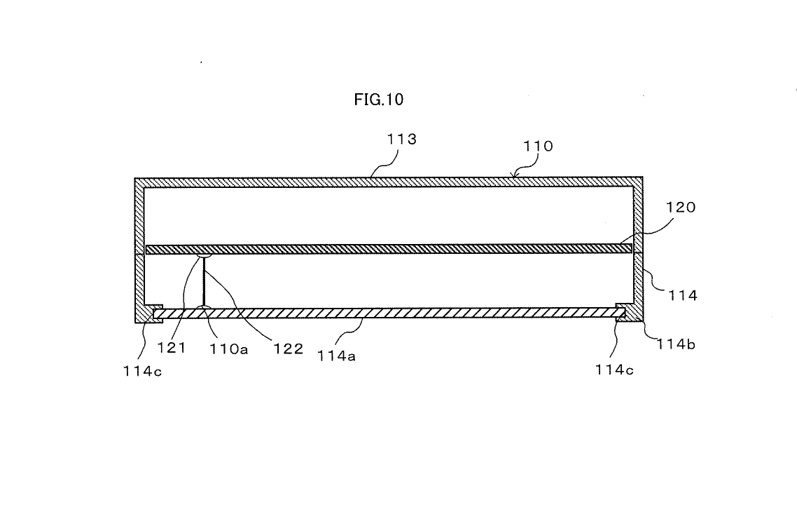

FIG. 8A and FIG. 8B are a front view and a rear view of the portable telephone 100 according to Embodiment 2 of the present invention, FIG. 9 is a exploded perspective view and FIG. 10 shows a cross-section along the cutting plane line B-B of FIG. 8A. The portable telephone 100 comprises the case 110 and the circuit board 120 housed within the case 110. The functional block diagram of the portable telephone according to Embodiment 2 and the circuit configuration of the biological information detector 170 according to Embodiment 2 are the same as those (examples shown in FIG. 3 and FIG. 5) in Embodiment 1.

The case 110 is formed in a boxlike shape with a front case 113 and a rear case 114. The front case 113 is formed from ABS (Acrylonitrile, Butadiene and Styrene) resin or from resin material of polycarbonate or acrylic or the like. The rear case 114 is integrally formed with the electrode 114a made of a tabular electroconductive material, and the resin part 114b made of resin material of ABS resin, polycarbonate or acrylic or the like.

The electrode 114a is set so that the surface exposed to the outside of the electrode 114a is roughly parallel to the surface of the circuit board 120. The resin part 114b encompasses the border of the electrode 114a and forms the side surface of the case 110.

When the user grasps the portable telephone 100, a part of the body (for example, the palm of the hand or the fingers or the like) makes contact with the exposed surface of the electrode 114a. More specifically, as shown in FIG. 10, the resin part 114b comprises, for example, a rectangular opening on a surface roughly parallel to the surface of the circuit board 120, and an engagements 114c roughly in the shape of “ko” in Japanese katakana character, the shape which resembles a character “U”, at the edge of the opening. The concave portions, roughly in the shape of “ko” in Japanese katakana character, of mutually opposing engagements 114c, are arranged to be mutually facing and opposing to each other, and the borders of the electrode 114a are engaged in the concave portions.

The rear case 114 is integrally formed with the electrode 114a and the resin part 114b by, for example, insert formation which is a method to form the resin part 114b by injecting resin into a mold after loading the electrode 114a in the mold. In addition, the rear case 114 may also be integrally formed with the electrode 114a and the resin part 114b by sticking them. The contact 110a is placed on the electrode 114a to which the contact 110a is conductive, and the contact 110a is electrically connected to the contact 121 on the circuit board 120 by the wire 122.

In the portable telephone 100 thus composed, the electrode 114a, which is a part of the case 110 functions as the electrode of the biological information detector 170. Accordingly, the biological information detector 170, as in aforementioned Embodiment 1, can take measurement for acquiring the biological information of the user touching the electrode 114a when the portable telephone 100 is operated by the user.

In the portable telephone 100 of Embodiment 2 composed as described above, the electrode 114a is formed in a part of the case 110 as the electrode to be used in measurement for acquiring the biological information. Therefore, even when the operation for, for example, e-mail transmission/reception or telephone conversations, or the like, of which the primary objective is not for acquiring biological information, the case 110 is grasped by the user, bringing the user into contact with the electrode 114a. Accordingly, as in the case of Embodiment 1, the portable telephone 100 is able to acquire the biological information of the user, even if there is no active action by the user to acquire the biological information.

In addition, in the portable telephone 100 according to Embodiment 2, portions of other than the electrode 114a of case 110 are formed from resin material. In other words, since resin material may also be used as the material of the case 110, by increased option of material selection, there is a lot of flexibility in the design of the portable telephone 100.

In addition, the contact 110a may be positioned anywhere on the surface of the electrode 114a, facing the surface of the circuit board 120, or in other words, anywhere on the inner surface of the electrode 114a. As a result, since there is a lot of flexibility in the arrangement of the contact 121 on the circuit board 120, for the contact with the electrode 114a, there is also a lot of flexibility in the design of the circuit board 120.

Note that the present invention is not limited to aforementioned Embodiment 1 and Embodiment 2, but various other modifications and applications of the present invention are possible.

For example, in Embodiment 1 and Embodiment 2, the contact 121 of the circuit board 120 is electrically connected to the case 110 or the contact 110a of the electrode 114a by the wire 122. However, the method of connection is not limited to these examples. For example, in Embodiment 1 and Embodiment 2, the contact 121 and the contact 110a are electrically connected by a single wire 122, but may also be electrically connected by plural wires. In addition, for example, the wire connecting between the contacts may also be replaced by a compression spring formed of electrically conductive material. The compression spring exerts elastic force on the contact 121 and on the case 110 or the electrode 114a, by being compressed in a state in which the circuit board 120 is housed within the case 110. Since this means that the work to fix the wire 122 to the contact 121 and to the case 110 or the electrode 114a by soldering becomes unnecessary, the efficiency of assembling the portable telephone 100 is improved.

In addition, in Embodiment 1 and Embodiment 2, an explanation is provided of a case in which the portable telephone 100 comprises a single case 110; however, it may also comprise plural cases. For example, in the case of comprising two cases, such as with a folding handset, at least one of the two cases may be composed of either of the cases of Embodiment 1 or Embodiment 2.

In addition, in Embodiment 2, the electrode 114a is provided across roughly the entire region of a part formed on a tabular shape of the rear case 114, roughly parallel to the surface of the circuit board 120; however, the location in which the electrode 114a is provided, and the breadth of the electrode 114a are not limited to these examples. For example, the electrode 114a may also be attached to the front case 113, or to a part of the surfaces roughly parallel to the surface of the circuit board 120.

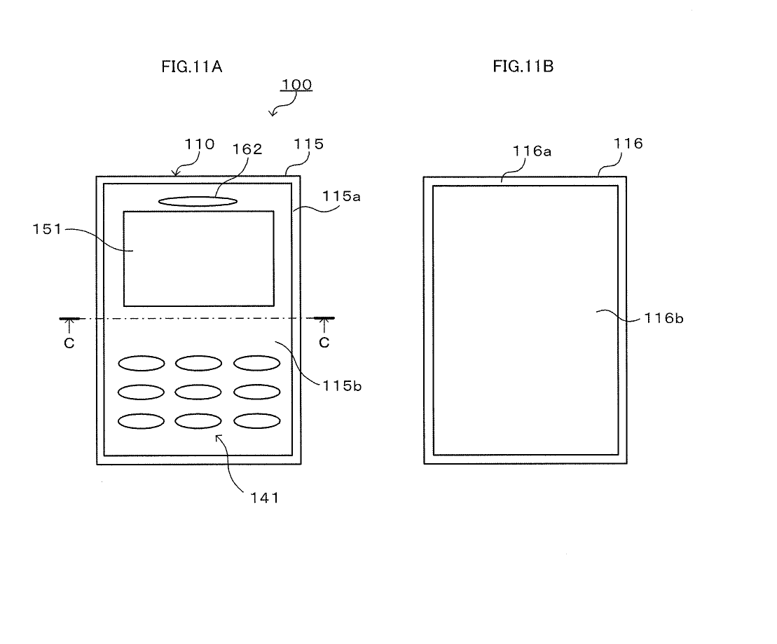

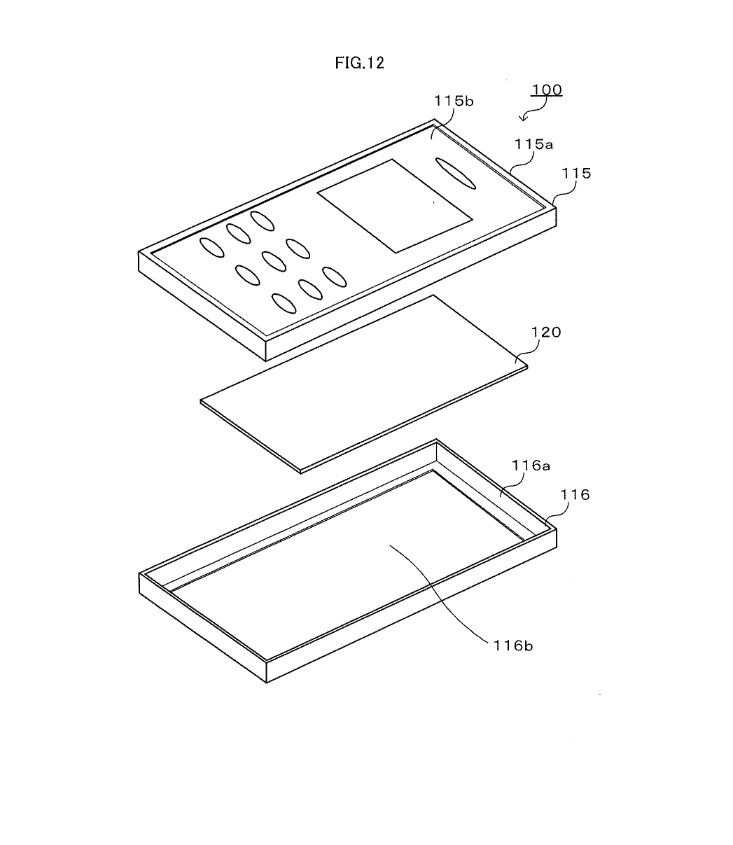

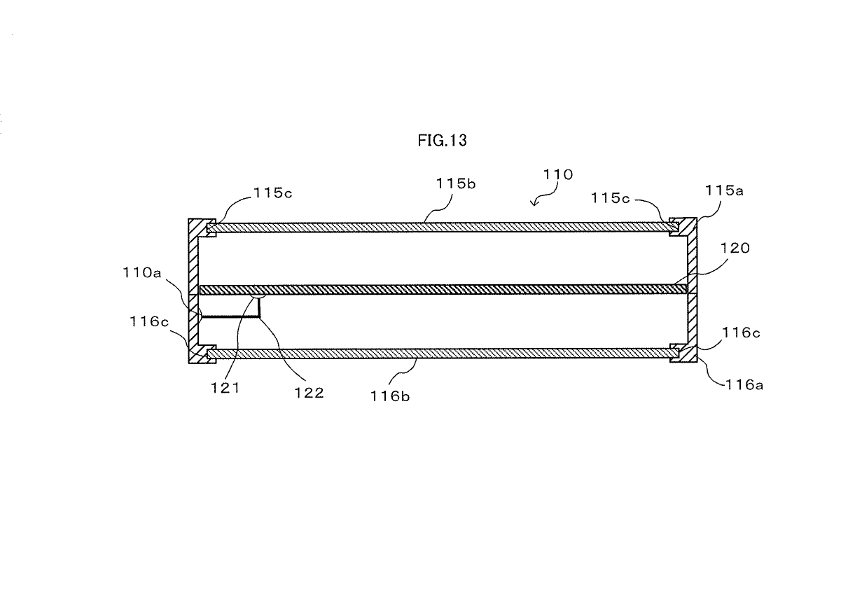

In addition, the electrode 114a may also be attached so as to face the side surface (border) of the circuit board 120, or in other words, may also be attached to the side surface of the case 110. FIG. 11A, FIG. 11B, FIG. 12 and FIG. 13 show examples of a portable telephone 100 in which the electrodes 115a and 116a are provided on the side surface of the case 110.

As shown in the figures, in a front case 115 and a rear case 116, the resin parts 115b and 116b, formed to be tabular, are provided roughly parallel to the surface of the circuit board 120. The electrodes 115a and 116a are attached so as to encompass the border of the resin parts 115b and 116b, forming the side surface of the case 110.

More specifically, as shown in FIG. 13, the electrodes 115a and 116a comprise the openings on a surface roughly parallel to the circuit board 120, and the engagements 115c and 116c which have the cross-sections roughly in the shape of “ko” in Japanese katakana character at the edges of the openings. The concave portions roughly in the shape of “ko” in Japanese katakana character, of engagements 115c and 116c are arranged facing each other, and the border of the concave portion of the engagement 115c and the border of the resin part 115b, as well as the concave portion of the engagement 116c and the border of the resin part 116b engage with each other respectively. In addition, the contact 110a on the electrode 116a is electrically connected to the contact 121 of the circuit board 120 by the wire 122. As shown in FIG. 13, the electrodes 115a and 116a are equipotential as mutually abutting at the ends of the opposite sides to the engagements 115c and 116c.

Accordingly, the electrodes 115a and 116a function in the same manner as the electrode 114a of Embodiment 1.

Specifically, the electrode 114a is provided, for example, in the region adjacent to the hardware keys 141 among the side surfaces of the case 110. Generally speaking, the user often uses the thumb of the right-hand (or the left-hand) with the region adjacent to the hardware keys 141 among the side surfaces of the case 110 being touched by the palm of the right-hand (or left-hand), the middle finger, the ring finger and the little finger when operating the hardware keys 141. Accordingly, with providing the electrode 114a in the region mentioned above, when the user operates the hardware keys 141, for example, to begin creating an e-mail, the controller 180 commences the biological information acquisition process. By this procedure, the user touches the electrode 114a by the hand while creating the e-mail, in other words, while operating the hardware keys 141. Accordingly, the portable telephone 100 is able to acquire the biological information of the user, even if there is no active action of the user to acquire the biological information.

In this manner, by providing the electrodes 115a and 116a on the side surfaces of the case 110, the portable telephone 100 can acquire the biological information of the user while the user is grasping the side surface of the case 110, even if the primary objective of the operation differs from the objective to acquire the biological information, for example, the operation of transmitting/receiving e-mail or having telephone communication, or the like, in other words, there is no active action of the user to acquire the biological information.

Note that the electrode may also be composed of plural electrodes which are physically separated each other. In this case, the plural electrodes are electrically connected by the wire or the like. Accordingly, since the plural electrodes are mutually in an equipotential state, the electrodes function as an electrode, and have the same effect as the electrode 114a has in Embodiment 1.

Embodiments may be modified as long as there is no deviation from the broad meaning or scope of the present invention. The aforementioned embodiments are not intended to restrict the scope of the present invention, but to explain the present invention. The scope of the present invention, more than the embodiments, is explained by each claim of the attached Scope of Patent Claims. Various modification of the invention accomplished within the scope equivalent to that of each claim is included within the scope of the present invention.

This application claims the benefit of Japanese Patent Application 2010-075965 filed Mar. 29, 2010. The entire specification, claims, and drawings of Japanese Patent Application 2010-075965 are incorporated herein by reference in this specification.

### DESCRIPTION OF REFERENCE NUMERALS

100 Portable telephone

110 Case

110a Contact

111 Front case

112 Rear case

113 Front case

114 Rear case

114a Electrode

114b Resin part

114c Engagement

115 Front case

115a Electrode

115b Resin part

115c Engagement

116 Rear case

116a Electrode

116b Resin part

116c Engagement

120 Circuit board

121 Contact

122 Wire

130 Communicator

131 Internal antenna

140 Inputter

141 Hardware keys

150 Display

151 Display panel

160 Audio processor

161 Microphone

162 Speaker

170 Biological information detector

171 Oscillator

172 Passive element

173 Voltage measurement circuit

180 Controller

181 CPU

182 ROM

183 RAM

184 Input/output I/F

1. A portable terminal device comprising:
a case that comprises an electrode which has a surface exposed to the outside, on at least a part of the case, wherein the case includes:
an oscillator which outputs an A.C. voltage signal;
a passive element, one end of which is connected to the oscillator, and the other end of which is connected to the electrode;
a voltage measurement circuit, which detects the voltage applied to the passive element by being connected to both ends of a passive element; and
a controller which acquires the biological information of a living body, based on a measured voltage value comprising the voltage detected by the voltage measurement circuit in a state in which an A.C. voltage signal is output from the oscillator, and in which the living body is in contact with the surface exposed to the outside of the electrode.

2. The portable terminal device according to claim 1, wherein the case entirely comprises an electrode, entire outer surfaces of the electrode being exposed to an outside.

3. The portable terminal device according to claim 1, wherein:
the electrode comprises plural electrodes which are separated, and electrically connected each other, and the outer surfaces of the electrodes are exposed to the outside.

4. The portable terminal device according to claim 1, further comprising a circuit board arranged within the case and provided with contacts electrically connected to the electrode and the passive element respectively, wherein
the electrode comprises a surface exposed to the outside, which is parallel to the surface of the circuit board.

5. The portable terminal device according to claim 1 further comprising a circuit board arranged within the case, and provided with contacts electrically connected to the electrode and the passive element respectively, wherein
the electrode comprises a surface exposed to the outside, parallel to the side surface of the circuit board.

6. The portable terminal device according to claim 1, wherein the case comprises a resin part.

7. The portable terminal device according to claim 1, comprising a storage, wherein:
the storage stores the voltage, as the standard voltage value, detected by the voltage measurement circuit in a state in which an A.C. voltage signal is output from the oscillator and the living body is not connected to the surface exposed to the outside of the electrode; and
the controller calculates the difference between the measured voltage value and the standard voltage value as the differential voltage value, and acquires the biological information based on the differential voltage value.

8. The portable terminal device according to claim 7, wherein:
the storage stores correlation information that is the information showing the relationship between the differential voltage value and the biological information; and
the controller acquires the biological information based on the correlation information and the differential voltage value.

9. A biological information acquisition method executed with a portable terminal device, comprising:
an A.C. voltage output step of outputting A.C. voltage from an oscillator;
a voltage detection step of detecting a voltage applied to a passive element by the A.C. voltage output in the A.C. voltage output step, wherein one end of the passive element is connected to the oscillator, and the other end is connected to an electrode, in a state in which a living body makes contact with the electrode which has a surface exposed to the outside, the electrode which is provided on at least a part of a case of the portable terminal device; and
a biological information acquisition step of acquiring the biological information of a living body based on the voltage detected in the voltage detection step.

10. The biological information acquisition method according to claim 9, wherein:
in the voltage detection step, detecting the voltage applied to the passive element is executed with being triggered by an operation which includes an objective other than the detection of the biological information as a primary objective.
